(19) United States
(12) Patent Application Publication
(10) Pub. No.: US 2022/0319687 A1
(43) Pub. Date: Oct. 6, 2022

(54) CONTROL METHOD OF BONE GRINDING, TERMINAL DEVICE AND STORAGE MEDIUM

(71) Applicant: BOE TECHNOLOGY GROUP CO., LTD., Beijing (CN)

(72) Inventors: Hui WEI, Beijing (CN); Guizhen LIU, Beijing (CN)

(73) Assignee: BOE TECHNOLOGY GROUP CO., LTD., Beijing (CN)

(21) Appl. No.: 17/620,367

(22) PCT Filed: Feb. 2, 2021
(86) PCT No.: PCT/CN2021/074808

§ 371 Date: Dec. 17, 2021

(30) Foreign Application Priority Data
Feb. 21, 2020 (CN) 2020 10107328.9

(51) Int. Cl. G16H 40/63; A61F 2/46; A61B 17/16; A61B 90/00

(52) U.S. Cl. CPC G16H 40/63; A61F 2/4618; A61B 17/1675; A61B 90/37; A61B 2090/061; A61B 2017/00022

(57) ABSTRACT

A control method of bone grinding includes: receiving a current position of a grinding drill bit from a positioning apparatus in real time; determining whether the current position reaches a region boundary of a region to be ground in a grinding state; and controlling the grinding drill bit to stop operating when the current position reaches the region boundary.

### CROSS-REFERENCE TO RELATED APPLICATIONS

This application is a national phase entry under 35 USC 371 of International Patent Application No. PCT/CN 2021/074808 filed on Feb. 2, 2021, which claims priority to Chinese Application No. 202010107328.9, filed on Feb. 21, 2020, which are incorporated herein by reference in their entirety.

### TECHNICAL FIELD

The present disclosure relates to the field of data processing technologies, and in particular, to a control method of bone grinding, a terminal device and a storage medium.

### BACKGROUND

During a normal joint movement, cartilage in the joint may be effective in reducing friction between bones. When a disease such as osteoarthritis occurs, the cartilage is aged or deformed, and thus the friction between bones is easily increased, which causes joint swelling, pain and bandy legs.

At present, the friction between bones may be replaced by friction between metal and plastic through a knee replacement, so that symptoms of a patient are relieved, joint function is improved, and life quality is improved. Here, the knee replacement is to remove the aged cartilage by grinding, and install a metal prosthesis and a wear-resistant plastic spacer on a trimmed bone surface.

### SUMMARY

In an aspect, a control method of bone grinding is provided. The control method of bone grinding includes: receiving a current position of a grinding drill bit from a positioning apparatus in real time; determining whether the current position reaches a region boundary of a region to be ground in a grinding state; and controlling the grinding drill bit to stop operating when the current position reaches the region boundary.

In some embodiments, determining whether the current position reaches the region boundary of the region to be ground, includes: selecting a plurality of position points on the region boundary as reference position points; calculating a distance between each reference position point and the current position; and determining that the current position reaches the region boundary when there is a distance, between at least one reference position point and the current position, that is substantially zero.

In some embodiments, along the region boundary, a distance between every two adjacent reference position points is substantially equal to a set step value.

In some embodiments, after calculating the distance between the current position and each reference position point, the control method further includes: determining whether there is a distance, between at least one reference position point and the current position, that is less than or equal to a preset critical value; and if there is the distance, between the at least one reference position point and the current position, that is less than or equal to the preset critical value, sending a prompt control signal to a terminal device, so as to control the terminal device to send out a prompt signal for prompting that the grinding drill bit is adjacent to the region boundary.

In some embodiments, there are a plurality of preset critical values, and the plurality of preset critical values are unequal. Determining whether there is the distance, between the at least one reference position point and the current position, that is less than or equal to the preset critical value, includes: performing a plurality of determinations according to the plurality of preset critical values, respectively. Each determining process is to determine whether there is a distance, between at least one reference position point and the current position, that is less than or equal to a corresponding preset critical value. When a determination result indicates that there is a distance, between at least one reference position point and the current position, that is less than or equal to a corresponding preset critical value, a different prompt control signal is sent correspondingly, so as to control the terminal device to send out a different prompt signal.

In some embodiments, the plurality of determinations performed respectively according to the plurality of preset critical values are successively performed in a descending order of the plurality of preset critical values.

In some embodiments, when there is the distance, between the at least one reference position point and the current position, that is less than or equal to the preset critical value, a deceleration control signal is sent to the grinding drill bit, so as to control an operating speed of the grinding drill bit to be decreased. In some embodiments, when there are a plurality of preset critical values, and the plurality of preset critical values are unequal, if a determination result indicates that there is a distance, between at least one reference position point and the current position, that is less than or equal to a corresponding preset critical value, a different deceleration control signal is sent correspondingly, so as to control the operating speed of the grinding drill bit to be decreased to a preset operating speed. The preset operating speed decreases as the preset critical value decreases.

In some embodiments, the plurality of preset critical values include a first preset critical value and a second preset critical value, and the second preset critical value is less than the first preset critical value. Performing the plurality of determinations according to the plurality of preset critical values, respectively; and when the determination result indicates that there is the distance, between the at least one reference position point and the current position, that is less than or equal to the corresponding preset critical value, sending the different prompt control signal correspondingly, include: determining whether there is a distance, between at least one reference position point and the current position, that is less than or equal to the first preset critical value; if there is the distance, between the at least one reference position point and the current position, that is less than or equal to the first preset critical value, sending a first prompt control signal to the terminal device, so as to control the terminal device to send out a first prompt signal for prompting that the grinding drill bit is adjacent to the region boundary; at a next moment, determining whether there is a distance, between at least one reference position point and a current position corresponding to the next moment, that is less than or equal to the second preset critical value; and if there is the distance, between the at least one reference position point and the current position corresponding to the next moment, that is less than or equal to the second preset critical value, sending a second prompt control signal to the terminal device, so as to control the terminal device to send out a second prompt signal for prompting that the grinding drill bit is adjacent to the region boundary.

In some embodiments, the control method further includes: when there is the distance, between the at least one reference position point and the current position, that is less than or equal to the first preset critical value, sending a first deceleration control signal to the grinding drill bit, so as to control an operating speed of the grinding drill bit to be decreased to a first preset operating speed; and when there is the distance, between the at least one reference position point and the current position corresponding to the next moment, that is less than or equal to the second preset critical value, sending a second deceleration control signal to the grinding drill bit, so as to control the operating speed of the grinding drill bit to be decreased to a second preset operating speed. The second preset operating speed is less than the first preset operating speed.

In some embodiments, the plurality of preset critical values further include a third preset critical value, and the third preset critical value is less than the second preset critical value. Performing the plurality of determinations according to the plurality of preset critical values, respectively; and when the determination result indicates that there is the distance, between the at least one reference position point and the current position, that is less than or equal to the corresponding preset critical value, sending the different prompt control signal correspondingly, further includes: when there is the distance, between the at least one reference position point and the current position, that is less than or equal to the second preset critical value, at a next moment, determining whether there is a distance, between at least one reference position point and a current position corresponding to the next moment, is less than or equal to the third preset critical value; and if there is the distance, between the at least one reference position point and the current position corresponding to the next moment, that is less than or equal to the third preset critical value, sending a third prompt control signal to the terminal device, so as to control the terminal device to send out a third prompt signal for prompting that the grinding drill bit is adjacent to the region boundary.

In some embodiments, when there is the distance, between the at least one reference position point and the current position corresponding to the next moment, that is less than or equal to the third preset critical value, a third deceleration control signal is sent to the grinding drill bit, so as to control the operating speed of the grinding drill bit to be decreased to a third preset operating speed. The third preset operating speed is less than the second preset operating speed.

In some embodiments, after controlling the grinding drill bit to stop operating, the control method further includes: determining whether the current position of the grinding drill bit is adjusted back into the region to be ground without being located on the region boundary of the region to be ground; if the current position of the grinding drill bit is adjusted back into the region to be ground without being located on the region boundary of the region to be ground, sending an in-position control signal to a terminal device, so as to control the terminal device to send out a prompt signal for prompting that the grinding drill bit is adjusted back into the region to be ground; and in response to an operation for restarting the grinding drill bit, controlling the grinding drill bit to restart.

In some embodiments, the control method further includes: in response to an operation for indicating a completed grinding, determining a ground region; and sending data of the region to be ground and data of the determined ground region to a display apparatus, so that the display apparatus generates a comparison image of the region to be ground and the ground region, and displays the comparison image.

In some embodiments, before receiving the current position of the grinding drill bit from the positioning apparatus in real time, the control method further includes: receiving image data of a portion to be ground from a medical imaging device; and determining a region to be ground corresponding to the portion to be ground and a region boundary of the region to be ground according to the image data.

In some embodiments, before determining whether the current position reaches the region boundary of the region to be ground, the control method further includes: determining whether the current position of the grinding drill bit is in the region to be ground; if the current position of the grinding drill bit is in the region to be ground, determining whether the current position reaches the region boundary of the region to be ground.

In another aspect, a terminal device is provided. The terminal device includes a second communication circuit, a main control chip and an alarm coupled to the main control chip. The second communication circuit is configured to receive a prompt control signal. The main control chip is further coupled to the second communication circuit and configured to, in response to the prompt control signal, control the alarm to send out a prompt signal for prompting that the grinding drill bit is adjacent to the region boundary of the region to be ground.

In some embodiments, the second communication circuit is further configured to receive information of the current position. The terminal device further includes a display screen coupled to the main control chip and configured to display the current position of the grinding drill bit in real time according to the information of the current position under a control of the main control chip.

In yet another aspect, a non-transitory computer-readable storage medium is provided, which stores computer program instructions that, when run on a processor, enable the processor to perform one or more steps of the control method in any one of the above embodiments.

In yet another aspect, a computer program product is provided. The computer program product includes computer program instructions that, when executed on a computer, enable the computer to perform one or more steps of the control method in any one of the above embodiments.

### BRIEF DESCRIPTION OF THE DRAWINGS

In order to describe technical solutions in the present disclosure more clearly, accompanying drawings to be used in some embodiments of the present disclosure will be introduced briefly below. Obviously, the accompanying drawings to be described below are merely accompanying drawings of some embodiments of the present disclosure, and a person of ordinary skill in the art may obtain other drawings based on these drawings. In addition, the accompanying drawings to be described below may be regarded as schematic diagrams, but are not limitations on an actual size of a product, an actual process of a method and an actual timing of a signal involved in the embodiments of the present disclosure.

FIG. 1 is a flow diagram of a control method of bone grinding, in accordance with some embodiments;

FIG. 2 is a flow diagram of another control method of bone grinding, in accordance with some embodiments;

FIG. 3 is a flow diagram of yet another control method of bone grinding, in accordance with some embodiments;

FIG. 4 is a flow diagram of yet another control method of bone grinding, in accordance with some embodiments;

FIG. 5 is a flow diagram of yet another control method of bone grinding, in accordance with some embodiments;

FIG. 6 is a flow diagram of yet another control method of bone grinding, in accordance with some embodiments;

FIG. 7 is a flow diagram of yet another control method of bone grinding, in accordance with some embodiments;

FIG. 8 is a flow diagram of yet another control method of bone grinding, in accordance with some embodiments;

FIG. 9 is a flow diagram of yet another control method of bone grinding, in accordance with some embodiments;

FIG. 10 is a flow diagram of yet another control method of bone grinding, in accordance with some embodiments;

FIG. 11 is a flow diagram of yet another control method of bone grinding, in accordance with some embodiments;

FIG. 12 is a structural diagram of a control apparatus applied to bone grinding, in accordance with some embodiments;

FIG. 13 is a structural diagram of another control apparatus applied to bone grinding, in accordance with some embodiments;

FIG. 14 is a structural diagram of yet another control apparatus applied to bone grinding, in accordance with some embodiments;

FIG. 15 is a structural diagram of a grinding device, in accordance with some embodiments;

FIG. 16 is a structural diagram of another grinding device, in accordance with some embodiments;

FIG. 17 is a structural diagram of a terminal device, in accordance with some embodiments;

FIG. 18 is a structural diagram of another terminal device, in accordance with some embodiments; and

FIG. 19 is a structural diagram of a grinding system, in accordance with some embodiments.

### DETAILED DESCRIPTION

Technical solutions in some embodiments of the present disclosure will be described clearly and completely below with reference to the accompanying drawings below. Obviously, the described embodiments are merely some but not all embodiments of the present disclosure. All other embodiments obtained by a person of ordinary skill in the art based on the embodiments of the present disclosure shall be included in the protection scope of the present disclosure.

Unless the context requires otherwise, throughout the description and the claims, the term “comprise” and other forms thereof such as the third-person singular form “comprises” and the present participle form “comprising” are construed as an open and inclusive meaning, i.e., “including, but not limited to”. In the description of the specification, the terms such as “one embodiment”, “some embodiments”, “exemplary embodiments”, “an example”, “specific example” or “some examples” are intended to indicate that specific features, structures, materials or characteristics related to the embodiment(s) or example(s) are included in at least one embodiment or example of the present disclosure. Schematic representations of the above terms do not necessarily refer to the same embodiment(s) or example(s). In addition, the specific features, structures, materials or characteristics may be included in any one or more embodiments or examples in any suitable manner.

Hereinafter, the terms such as “first” and “second” are only used for descriptive purposes, and are not to be construed as indicating or implying the relative importance or implicitly indicating the number of indicated technical features. Thus, a feature defined with “first” or “second” may explicitly or implicitly include one or more of the features. In the description of the embodiments of the present disclosure, the term “a plurality of/the plurality of” means two or more unless otherwise specified.

In the description of some embodiments, the terms “coupled” and “connected” and their derivatives may be used. For example, the term “connected” may be used in the description of some embodiments to indicate that two or more components are in direct physical or electrical contact with each other. For another example, the term “coupled” may be used in the description of some embodiments to indicate that two or more components are in direct physical or electrical contact. However, the term “coupled” or “communicatively coupled” may also mean that two or more components are not in direct contact with each other, but still cooperate or interact with each other. The embodiments disclosed herein are not necessarily limited to the contents herein.

The phrase “at least one of A, B and C” has a same meaning as the phrase “at least one of A, B or C”, both including the following combinations of A, B and C: only A, only B, only C, a combination of A and B, a combination of A and C, a combination of B and C, and a combination of A, B and C.

The phrase “A and/or B” includes the following three combinations: only A, only B, and a combination of A and B.

As used herein, the term “if” is optionally construed as “when” or “in a case where” or “in response to determining” or “in response to detecting”, depending on the context. Similarly, the phrase “if it is determined” or “if [a stated condition or event] is detected” is optionally construed as “in a case where it is determined” or “in response to determining” or “in a case where [the stated condition or event] is detected” or “in response to detecting [the stated condition or event]”, depending on the context.

The use of the phrase “configured to” herein means an open and inclusive language, which does not exclude devices that are applicable to or configured to perform additional tasks or steps.

In addition, the use of the phrase “based on” means openness and inclusiveness, since a process, step, calculation or other action that is “based on” one or more of the stated conditions or values may, in practice, be based on additional conditions or values exceeding those stated.

The term such as “about”, “substantially” or “approximately” as used herein includes a stated value and an average value within an acceptable range of deviation of a particular value determined by a person of ordinary skill in the art, considering measurement in question and errors associated with measurement of a particular quantity (i.e., limitations of a measurement system).

The Knee replacement is required to remove the aged cartilage by grinding, and install the metal prosthesis and the wear-resistant plastic spacer on the trimmed bone surface. When removing the aged cartilage by grinding, a doctor usually grinds the aged cartilage based on clinical experience. In this way, the aged cartilage is incompletely removed, or healthy cartilage is partially removed, and thus it is impossible to ensure that the aged cartilage is accurately removed.

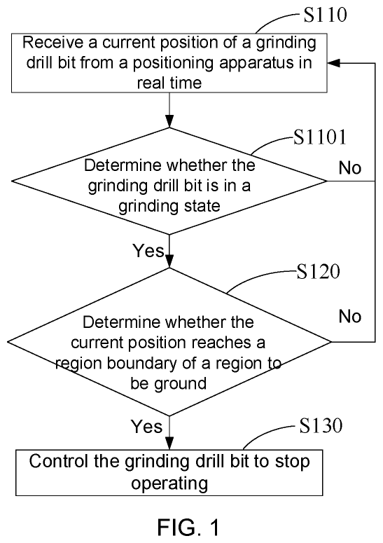

Based on this, in some embodiments of the present disclosure, a control method of bone grinding is provided. As shown in FIG. 1, the control method includes S110 to S130.

In S110, a current position of a grinding drill bit from a positioning apparatus is received in real time.

The current position is determined by the positioning apparatus based on a pre-established coordinate system. For example, the positioning apparatus is an optical navigation apparatus, and an optical coordinate system is established by taking the optical navigation apparatus or a base to which the grinding drill bit belongs as a reference. The current position is a real-time coordinate of a grinding end of the grinding drill bit in the optical coordinate system.

In S1101, it is determined whether the grinding drill bit is in a grinding state.

Whether the grinding drill bit is in the grinding state may be determined by determining whether the grinding drill bit is subjected to a resistance. For example, the grinding drill bit is in the grinding state when being subjected to the resistance, and the grinding drill bit is not in the grinding state when being not subjected to the resistance. Of course, whether the grinding drill bit is in the grinding state may also be determined according to other ways, which is not limited.

In the grinding state, S120, in which it is determined whether the current position reaches a region boundary of a region to be ground, is performed.

In addition, the region to be ground is a region where bone is required to be ground with the grinding drill bit. The region to be ground may include a diseased region of the bone and/or a partially normal region (facilitating the installation of the prosthesis) around the diseased region.

When the current position of the grinding drill bit reaches the region boundary, S130, in which the grinding drill bit is controlled to stop operating, is performed.

It can be seen from the above that in the control method of bone grinding in some embodiments of the present disclosure, in the grinding state, it is determined in real time whether the current position of the grinding drill bit reaches the region boundary of the region to be ground, and when the current position reaches the region boundary of the region to be ground, the grinding drill bit is controlled to stop operating. That is, during a process that the grinding drill bit grinds from an inside to an outside of the region to be ground in the grinding state, when reaching the region boundary of the region to be ground, the grinding drill bit automatically stops operating. In this way, a portion of the bone in the region to be ground may be completely removed, and bone outside the region boundary of the region to be ground cannot be removed by grinding. Therefore, the incomplete removal of the aged cartilage or the partial removal of the healthy cartilage may be effectively avoided, thereby ensuring that the portion of the bone in the region to be ground is accurately removed, and thus the grinding accuracy is improved.

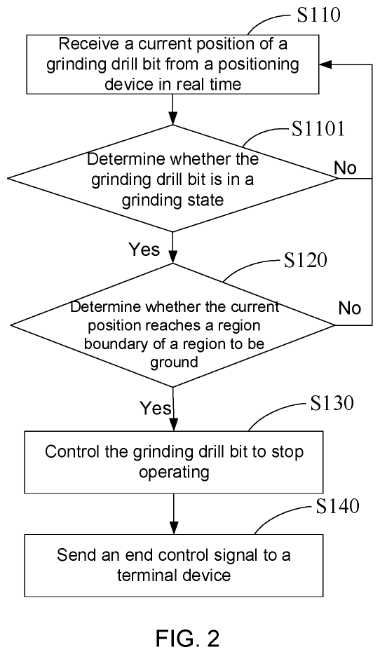

In some embodiments, referring to FIG. 2, after S130, the control method of bone grinding further includes S140.

In S140, an end control signal is sent to a terminal device. The end control signal may control the terminal device to send out a prompt signal for prompting an operator that the grinding drill bit stops operating.

Here, the prompt signal for prompting the operator that the grinding drill bit stops operating may be at least one of vibration, sound, and light generated by the terminal device, which is not limited. For example, the prompt signal may be light generated by the terminal device. For example, the prompt signal may be alternately flickering red and yellow light generated by the terminal device.

In this way, when the current position reaches the region boundary and the grinding drill bit stops operating, the terminal device sends the prompt signal for prompting the operator that the grinding drill bit stops operating, which may promptly remind the operator to perform subsequent operations.

Since the region boundary is generally an irregular curve, in order to determine whether the current position reaches the region boundary of the region to be ground, in some embodiments, a plurality of position points on the region boundary may be selected as reference position points, and whether the current position reaches the region boundary of the region to be ground is determined by determining whether there is a distance, between the reference position point and the current position, that is substantially zero.

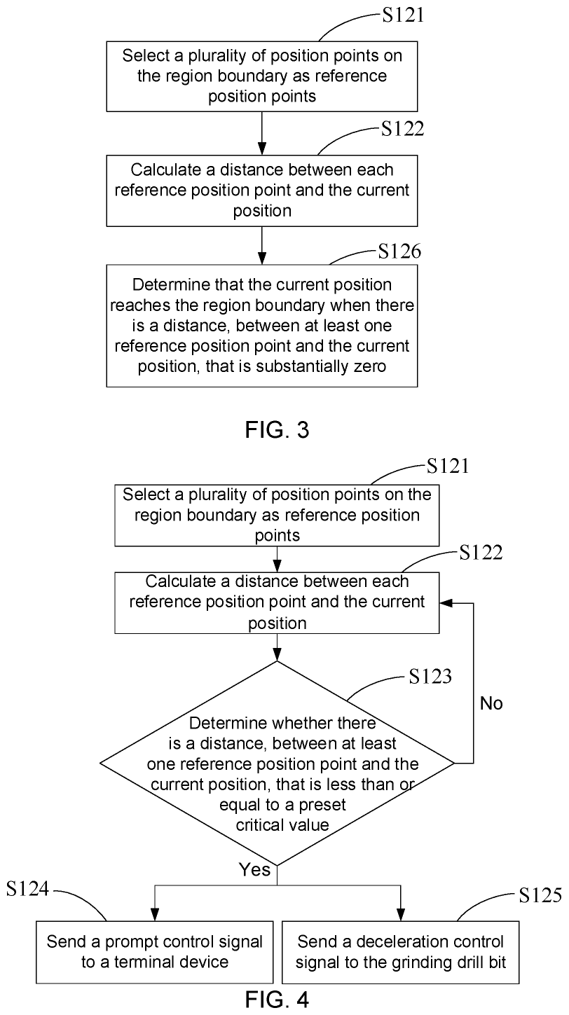

As shown in FIG. 3, S120 may include, for example, following steps.

In S121, the plurality of position points on the region boundary are selected as the reference position points.

For example, along the region boundary, a distance between every two adjacent reference position points is substantially equal to a set step value.

Here, a magnitude of the set step value is not limited. For example, the set step value may be 0.5 mm to 1.5 mm. For example, the set step value is 0.5 mm, 1 mm or 1.5 mm. In addition, the set step value may be adjusted based on a size of the region boundary. Of course, the set step value may also be adjusted according to other ways, which is not limited.

In S122, the distance between each reference position point and the current position is calculated.

Here, each reference position point and the current position are located in the same coordinate system. Therefore, the distance between each reference position point and the current position may be calculated based on coordinates.

In S126, when there is the distance, between at least one reference position point and the current position, that is substantially zero, it is determined that the current position reaches the region boundary.

Here, the distance between the reference position point and the current position is substantially zero, which may be that the distance between the current position outside the region to be ground and the reference position point is within an acceptable range of deviation, or may be that the distance between the current position within the region to be ground and the reference position point is within the acceptable range of deviation. For example, the current position is in the region to be ground, and the distance between the reference position point and the current position is less than 0.1 mm, then the distance between the reference position point and the current position is determined to be zero.

In some embodiments of the present disclosure, when the grinding drill bit is in the grinding state and adjacent to the region boundary of the region to be ground, the operator is prompted that the grinding drill bit is adjacent to the region boundary. In this way, the operator may prepare in advance for other work after the grinding drill bit stops according to prompt information that the grinding drill bit is adjacent to the region boundary, or may control a speed of the grinding drill bit to be decreased accordingly, so as to further improve grinding safety, and reduce a risk of grinding a region that is not required to be ground by the grinding drill bit, thereby well assisting the operator in grinding.

In some embodiments, referring to FIG. 4, after S122, the control method further includes S123 and S124.

In S123, it is determined whether there is a distance, between at least one reference position point and the current position, that is less than or equal to a preset critical value.

Here, a magnitude of the preset critical value is not limited. For example, the preset critical value may be 0.1 mm to 5 mm. For example, the preset critical value is 0.1 mm, 1 mm or 5 mm. In addition, the preset critical value may be adjusted based on a size of the region to be ground, which is not limited.

If yes, S124, in which a prompt control signal is sent to the terminal device, is performed. The prompt control signal may control the terminal device to send out a prompt signal for prompting the operator that the grinding drill bit is adjacent to the region boundary.

Here, the prompt signal for prompting the operator that the grinding drill bit is adjacent to the region boundary may be at least one of vibration, sound, and flickering light generated by the terminal device, which is not limited. For example, the prompt signal may be a sound generated by the terminal device. For example, the prompt signal may be a buzzing sound generated by the terminal device.

In some embodiments, after S123, referring to FIG. 4, the control method further includes:

if yes (i.e., if there is the distance, between the at least one reference position point and the current position, that is less than or equal to the preset critical value), performing S125, in which a deceleration control signal is sent to the grinding drill bit. The deceleration control signal is used to control an operating speed of the grinding drill bit to be decreased.

In some embodiments, there are a plurality of preset critical values, and the plurality of preset critical values are unequal. In this case, S123 may include, for example:

performing a plurality of determinations according to preset critical values, respectively. Each determining process is to determine whether there is a distance, between at least one reference position point and the current position, that is less than or equal to a corresponding preset critical value.

In this case, in a case where a determination result is yes, a different prompt control signals is sent correspondingly, so as to control the terminal device to send a different prompt signal for prompting the operator that the grinding drill bit is adjacent to the region boundary.

Similarly, in the case where a determinations result is yes, a different deceleration control signal is sent correspondingly, so as to control the operating speed of the grinding drill bit to be decreased to a corresponding preset operating speed. The preset operating speed decreases as the preset critical value decreases.

On this basis, the plurality of determinations performed respectively according to the plurality of preset critical values are successively performed in a descending order of the plurality of preset critical values. In a case where a determination is performed according a larger preset critical value, and the determination result is yes, a next determination is performed according to a smaller preset critical value.

In this case, for different preset critical values, the terminal device may send out different prompt signals at levels, which may well assist the operator in grinding. In addition, the operating speed of the grinding drill bit may also decrease as the preset critical value decreases, so as to further improve the grinding safety, and reduce the risk of grinding the region that is not required to be ground by the grinding drill bit.

For example, the preset critical value includes a first preset critical value and a second preset critical value, and the second preset critical value is less than the first preset critical value.

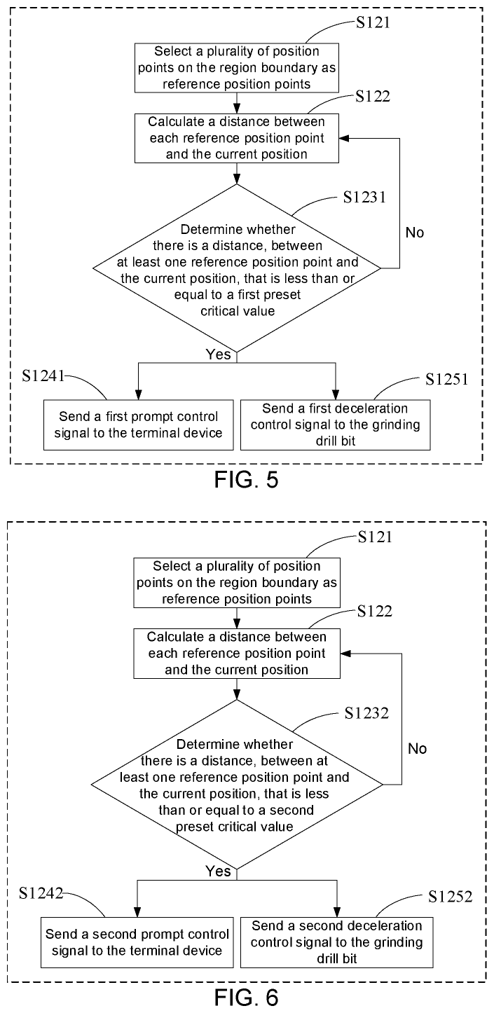

In this case, as shown in FIG. 5, S123 includes S1231, S124 includes S1241, and S125 includes S1251.

In S1231, it is determined whether there is a distance, between at least one reference position point and the current position, that is less than or equal to the first preset critical value.

If yes, S1241, in which a first prompt control signal is sent to the terminal device, is performed. The first prompt control signal may control the terminal device to send out a first prompt signal for prompting the operator that the grinding drill bit is adjacent to the region boundary.

S1251, in which a first deceleration control signal is sent to the grinding drill bit, may be performed synchronously. The first deceleration control signal is used to control the operating speed of the grinding drill bit to be decreased to a first preset operating speed.

As shown in FIG. 6, at a next moment, it is necessary to reacquire a current position and calculate a distance between each reference position point and the current position. The reference position point may be reselected on the region boundary, or the reference position point may be unchanged. Here, the next moment is a moment after S1231 is performed and the determination result is yes.

In this case, S123 includes S1232, S124 includes S1242, and S125 includes S1252.

In S1232, it is determined whether there is a distance, between at least one reference position point and the current position corresponding to the next moment, that is less than or equal to the second preset critical value.

If yes, S1242, in which a second prompt control signal is sent to the terminal device, is performed. The second prompt control signal may control the terminal device to send out a second prompt signal for prompting the operator that the grinding drill bit is adjacent to the region boundary.

S1252, in which a second deceleration control signal is sent to the grinding drill bit, may be performed synchronously. The second deceleration control signal is used to control the operating speed of the grinding drill bit to be decreased to a second preset operating speed.

Here, magnitudes of the first preset critical value and the second preset critical value are not limited, and the second preset critical value is less than the first preset critical value. For example, the first preset critical value may be 5 mm, and the second preset critical value may be 1 mm.

Here, magnitudes of the first preset operating speed and the second preset operating speed are not limited, and the second preset operating speed is less than the first preset operating speed. For example, a ratio of the first preset operating speed to an operating speed of the grinding drill bit operating at full power is 0.5, and a ratio of the second preset operating speed to the operating speed of the grinding drill bit operating at full power is 0.3.

Similarly, the first prompt signal and the second prompt signal are not limited, and the first prompt signal and the second prompt signal are different. For example, the first prompt signal and the second prompt signal both are buzzing sounds generated by the terminal device, and a volume of the buzzing sound generated by the terminal device corresponding to the first prompt signal is less than a volume of the buzzing sound generated by the terminal device corresponding to the second prompt signal.

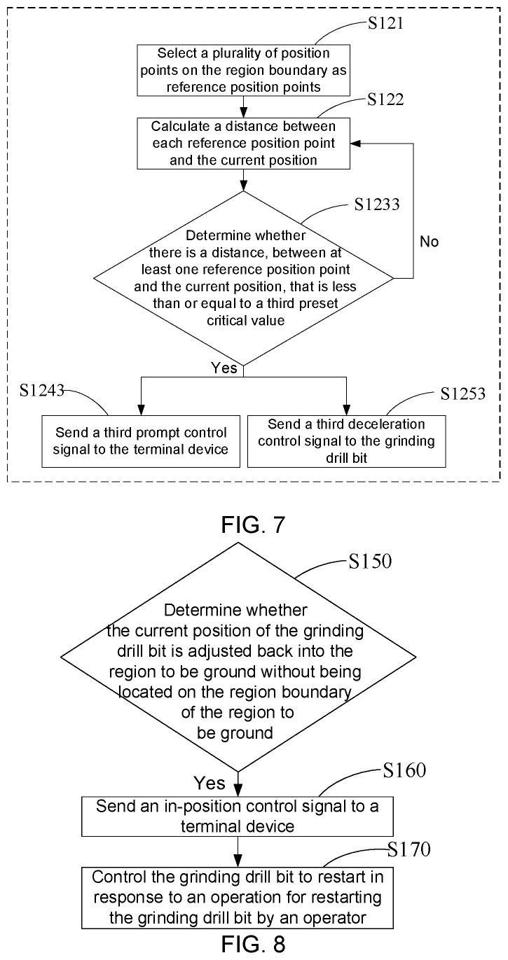

On this basis, in some embodiments, the preset critical value further includes a third preset critical value, and the third preset critical value is less than the second preset critical value. In this way, in a case where there is the distance, between the at least one reference position point and the current position, that is less than or equal to the second preset critical value, at a next moment, as shown in FIG. 7, a current position is reacquired, and a distance between each reference position point and the current position is calculated. Here, the next moment is a moment after S1231 is performed and the determination result is yes.

In this case, for example, S123 may further include S1233, S124 may further include S1243, and S125 may further include S1253.

In S1233, it is determined whether there is a distance, between at least one reference position point and the current position corresponding to the next moment, that is less than or equal to the third preset critical value.

If it is determined that there is the distance, between the at least one reference position point and the current position corresponding to the next moment, that is less than or equal to the third preset critical value, S1243, in which a third prompt control signal is sent to the terminal device, is performed. The third prompt control signal may control the terminal device to send out a third prompt signal for prompting the operator that the grinding drill bit is adjacent to the region boundary.

S1253, in which a third deceleration control signal is sent to the grinding drill bit, may be performed synchronously. The third deceleration control signal is used to control the operating speed of the grinding drill bit to be decreased to a third preset operating speed.

Here, a magnitude of the third preset critical value is not limited, and the third preset critical value is less than the second preset critical value. For example, the second preset critical value may be 1 mm, and the third preset critical value may be 0.1 mm.

Here, a magnitude of the third preset operating speed is not limited, and the third preset operating speed is less than the second preset operating speed. For example, a ratio of the second preset operating speed to the operating speed of the grinding drill bit operating at full power is 0.3, and a ratio of the third preset operating speed to the operating speed of the grinding drill bit operating at full power is 0.1.

Similarly, the third prompt signal is not limited, and the third prompt signal is different from the first prompt signal and the second prompt signal. For example, the first prompt signal, the second prompt signal and the third prompt signal are buzzing sounds generated by the terminal device. The volume of the buzzing sound generated by the terminal device corresponding to the first prompt signal is less than the volume of the buzzing sound generated by the terminal device corresponding to the second prompt signal, and the volume of the buzzing sound generated by the terminal device corresponding to the second prompt signal is less than a volume of the buzzing sound generated by the terminal device corresponding to the third prompt signal.

After S130, the grinding operation may not be completed, and the grinding drill bit is further required to be adjusted to another position in the region to be ground to continue grinding.

In some embodiments, as shown in FIG. 8, after S130, the control method further includes S150 to S170.

In S150, it is determined whether the current position of the grinding drill bit is adjusted back into the region to be ground without being located on the region boundary of the region to be ground.

If yes, S160, in which an in-position control signal is sent to the terminal device, is performed. The in-position control signal may control the terminal device to send out a prompt signal for prompting the operator that the grinding drill bit is adjusted back into the region to be ground.

Here, the prompt signal for prompting the operator that the grinding drill bit is adjusted back into the region to be ground may be at least one of vibration, sound, and flickering light generated by the terminal device, which is not limited. For example, the prompt signal may be a vibration generated by the terminal device. For example, the prompt signal may be a continuous vibration generated by the terminal device.

In S170, in response to an operation for restarting the grinding drill bit by the operator, the grinding drill bit is controlled to restart.

Of course, S150 to S170 may also be after S140, which is not limited.

In some embodiments, after a preset time interval of S130 or S140, S150 to S170 are automatically performed. In some other embodiments, after S130 or S140, in response to the operation for restarting the grinding drill bit by the operator, S150 is performed. In a case where the determination result of S150 is yes, the in-position control signal is sent to the terminal device, and the grinding drill bit is automatically controlled to restart in response to the in-position control signal. In yet other embodiments, after S130 or S140, in response to the operation for restarting the grinding drill bit by the operator, S150 to S170 are performed again. Here, the control of the restart of the grinding drill bit requires responding to the operation for restarting the grinding drill bit by the operator for two times. The first time is that S150 is performed in response to the operation for restarting the grinding drill bit by the operator, and the second time is that the grinding drill bit is controlled to restart in S170 in response to the operation for restarting the grinding drill bit by the operator.

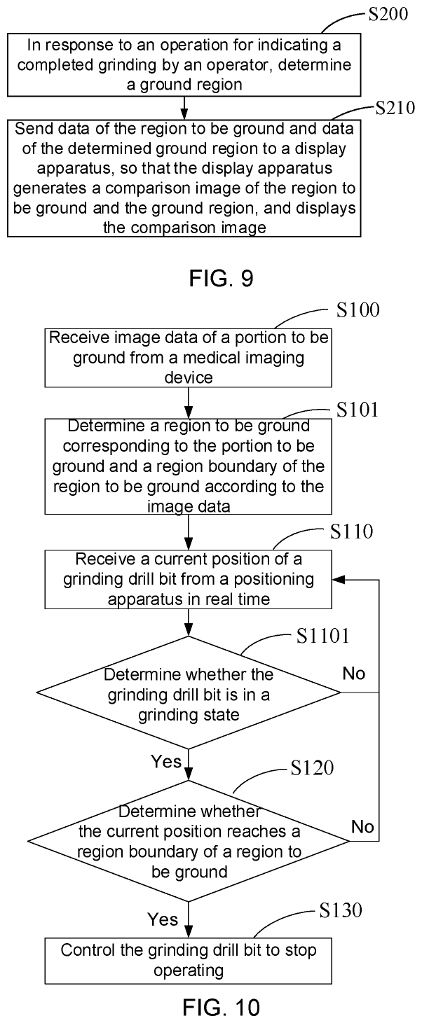

In some embodiments, as shown in FIG. 9, the control method further includes S200 and S210.

In S200, in response to an operation for indicating a completed grinding by the operator, a ground region is determined.

For example, the current positions through which the grinding drill bit passes in the region to be ground are combined to determine the ground region.

In S210, data of the region to be ground and data of the determined ground region are sent to a display apparatus, so that the display apparatus generates a comparison image of the region to be ground and the ground region, and displays the comparison image.

Here, the comparison image includes at least one of an image of a non-overlapped portion of the region to be ground and the ground region, an image of an overlapped portion of the region to be ground and the ground region, and an image for displaying the overlapped portion and the non-overlapped portion of the region to be ground and the ground region.

In this case, the generated comparison image may be displayed on the display apparatus, which enables the operator to know whether the region to be ground is completely ground clearly and intuitively.

In some embodiments, as shown in FIG. 10, before S110, the control method further includes S100 and S101.

In S100, image data of a portion to be ground is received from a medical imaging device.

Here, the image data includes a diseased feature and a diseased region at the portion to be ground.

In S101, a region to be ground corresponding to the portion to be ground and a region boundary of the region to be ground are determined according to the image data.

Here, the region to be ground is a region where bone is required to be ground with the grinding drill bit. The region to be ground may include a diseased region of the portion to be ground and/or a partially normal region (facilitating the installation of the prosthesis) around the diseased region. For example, according to the image data, the region to be ground is a diseased region of the bone. Of course, a doctor may also adjust the region to be ground based on clinical experience, which is not limited.

In addition, after the region to be ground is determined, the region boundary of the region to be ground is required to be determined. For example, the region boundary may be formed by all edge positions of the region to be ground, and the region boundary may be a closed line.

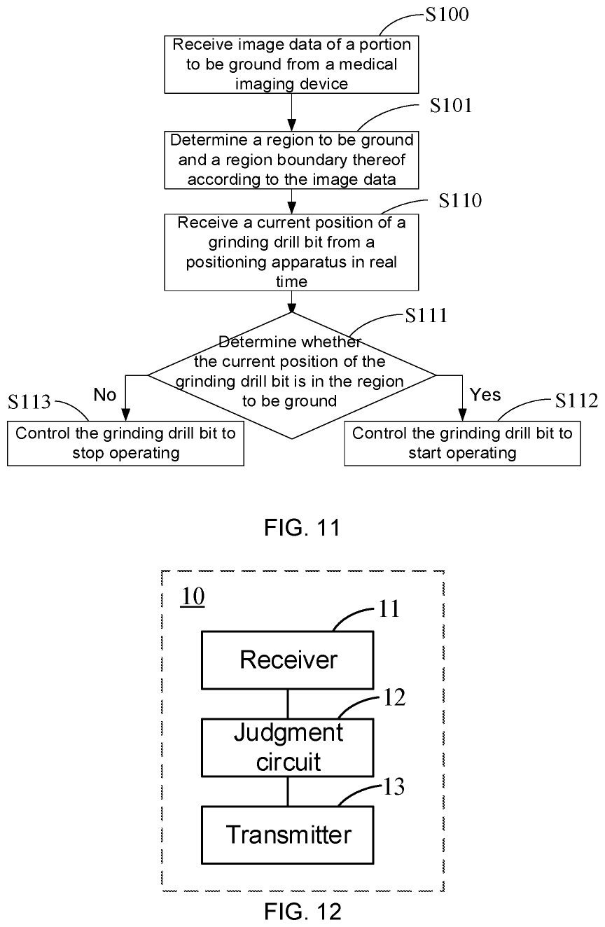

In some embodiments, before S120, referring to FIG. 11, the control method further includes S111 to S113.

In S111, it is determined whether the current position of the grinding drill bit is in the region to be ground.

If yes, S112, in which the grinding drill bit is controlled to start operating is performed.

That is, when the grinding drill bit is in the region to be ground, the grinding drill bit continues to operate. That is, the grinding drill bit is in the grinding state.

If no, S113, in which the grinding drill bit is controlled to stop operating, is performed.

For example, the region to be ground includes a plurality of positions to be ground, and both the current position and the positions to be ground are determined based on the pre-established coordinate system. The plurality of positions to be ground include all current positions of the grinding drill bit in the region to be ground. In this way, after the current position of the grinding drill bit is obtained, the current position is matched with each position to be ground in the region to be ground. If the position to be ground is overlapped with the current position, the current position is determined to be in the region to be ground. If the position to be ground is non-overlapped with the current position, the current position is determined not to be in the region to be ground. Of course, other ways may also be used to determine whether the current position is in the region to be ground, which is not limited.

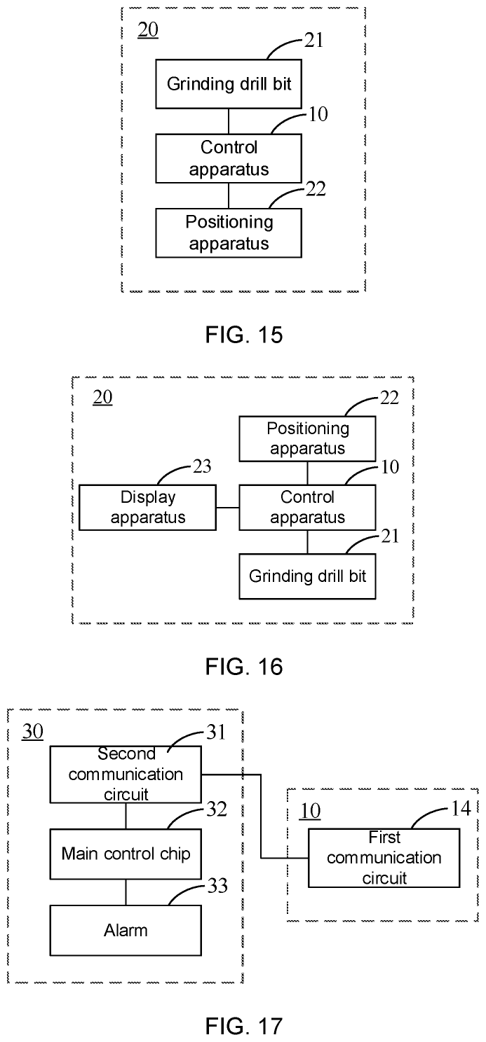

Referring to FIGS. 12 and 15, in some embodiments of the present disclosure, a control apparatus 10 applied to bone grinding is also provided. The control apparatus 10 includes a receiver 11, a determination circuit 12 and a transmitter 13. The receiver 11 is configured to be coupled to a positioning apparatus 22, so as to receive a current position of a grinding drill bit 21 from the positioning apparatus 22 in real time. The determination circuit 12 is configured to determine whether the current position reaches a region boundary of a region to be ground in a grinding state. The transmitter 13 is configured to be coupled to the grinding drill bit 21, and to send a control signal for controlling the grinding drill bit 21 to stop operating when the current position reaches the region boundary.

The technical problem solved by the control apparatus applied to bone grinding in some embodiments of the present disclosure is similar to the technical problem solved by the control method of bone grinding in the above embodiments, and thus will not be repeated here.

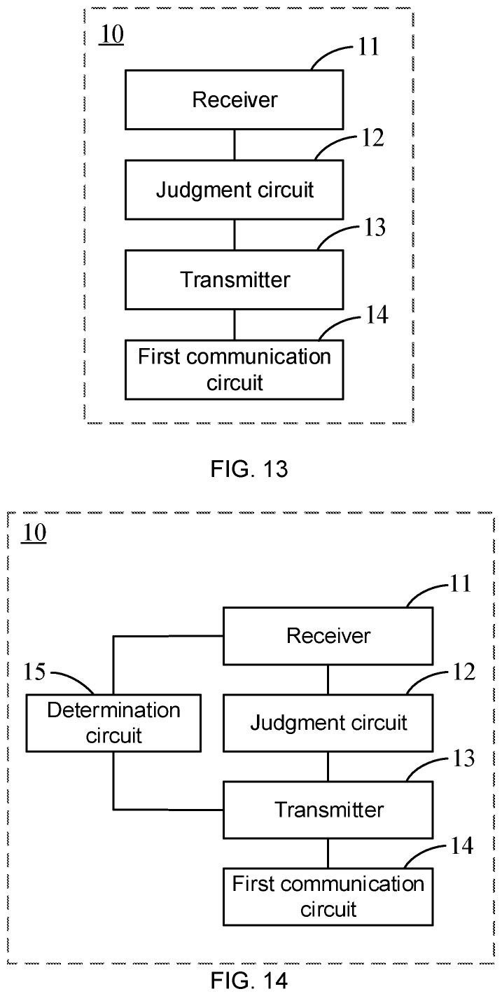

In some embodiments, referring to FIGS. 13, 14 and 19, the control apparatus 10 further includes a first communication circuit 14. The first communication circuit 14 is configured to send an end control signal to a terminal device 30, so as to control the terminal device 30 to send out a prompt signal for prompting an operator that the grinding drill bit 21 stops operating.

In this way, when the current position reaches the region boundary and the grinding drill bit 21 stops operating, the terminal device 30 may send out the prompt signal for prompting the operator that the grinding drill bit 21 stops operating, which may promptly remind the operator to perform subsequent operations.

Since the region boundary is generally an irregular curve, in order to determine whether the current position reaches the region boundary of the region to be ground, in some embodiments, a plurality of position points on the region boundary may be selected as reference position points, and whether the current position reaches the region boundary of the region to be ground is determined by determining whether there is a distance, between a reference position point and the current position, that is zero.

Based on this, as shown in FIG. 13, the determination circuit 12 is configured to: select a plurality of position points on the region boundary as reference position points; calculate a distance between each reference position point and the current position; determine whether there is a distance, between at least one reference position point and the current position, that is substantially zero; and if yes, determine that the current position reaches the region boundary. That is, the transmitter 13 is configured to, in a case where there is the distance, between the at least one reference position point and the current position, that is substantially zero, send the control signal for controlling the grinding drill bit 21 (refer to FIG. 15) to stop operating.

In addition, when the grinding drill bit 21 is in the grinding state and adjacent to the region boundary of the region to be ground, the operator may further be prompted that the grinding drill bit 21 is adjacent to the region boundary. In this way, the operator may prepare in advance for other work after the grinding drill bit stops according to prompt information that the grinding drill bit is adjacent to the region boundary, or may control a speed of the grinding drill bit to be decreased accordingly, so as to further improve grinding safety, and reduce a risk of grinding a region that is not required to be ground by the grinding drill bit, thereby well assisting the operator in grinding.

In some embodiments, as shown in FIG. 13, the determination circuit 12 is further configured to determine whether there is a distance, between at least one reference position point and the current position, that is less than or equal to a preset critical value. The control apparatus 10 includes the first communication circuit 14, and the first communication circuit 14 is configured to be coupled to the terminal device 30 (referring to FIG. 19), and to send a prompt control signal to the terminal device 30 in a case where there is the distance, between the at least one reference position point and the current position, that is less than or equal to the preset critical value, so as to control the terminal device 30 to send out a prompt signal for prompting the operator that the grinding drill bit 21 (referring to FIG. 15) is adjacent to the region boundary.

In this case, according to the prompt signal sent by the terminal device 30 for prompting the operator that the grinding drill bit 21 is adjacent to the region boundary, the operator may be well assisted in grinding.

In some embodiments, the transmitter 13 is further configured to, in the case where there is the distance, between the at least one reference position point and the current position, that is less than or equal to the preset critical value, send a deceleration control signal to the grinding drill bit 21 to control an operating speed of the grinding drill bit 21 to be decreased.

In this case, in the case where there is the distance, between the at least one reference position point and the current position, that is less than or equal to the preset critical value, the operating speed of the grinding drill bit 21 may also be decreased as the preset critical value decreases, so as to further improve the grinding safety, and reduce the risk of grinding the region that is not required to be ground by the grinding drill bit 21.

After the transmitter 13 sends the control signal for controlling the grinding drill bit 21 to stop operating to the grinding drill bit 21, the grinding operation may not be completed, and the grinding drill bit 21 is further required to be moved to another position in the region to be ground to continue grinding.

In some embodiments, the receiver 11 is further configured to receive the image data of the portion to be ground from the medical imaging device and a trigger signal for indicating a completed grinding by the operator. As shown in FIG. 14, the control apparatus 10 further includes a determination circuit 15 configured to determine a region to be ground corresponding to the portion to be ground and a region boundary of the region to be ground according to the image data received by the receiver 11 and determine a ground region in response to the trigger signal received by the receiver 11. The transmitter 13 is further configured to be coupled to a display apparatus 23 (referring to FIG. 16), and send data of the region to be ground and data of the ground region to the display apparatus 23.

In this case, a generated comparison image may be displayed on the display apparatus, which enables the operator to know whether the region to be ground is completely ground clearly and intuitively. In addition, the terminal device may further display the current position of the grinding drill bit in real time according to received information of the current position, so as to well assist the operator in grinding.

As shown in FIG. 15, in some embodiments of the present disclosure, a grinding device 20 is further provided. The grinding device 20 includes the control apparatus 10, the grinding drill bit 21 and the positioning apparatus 22 in any one of the above embodiments. The grinding drill bit 21 is coupled to the control apparatus 10, and the grinding drill bit 21 is configured to grind the region to be ground. The positioning apparatus 22 is disposed on the grinding drill bit 21, and is coupled to the control apparatus 10. The positioning apparatus 22 is configured to detect the current position of the grinding drill bit 21 in real time and send the detected current position to the control apparatus 10.

The technical problem solved by the grinding device 20 in some embodiments of the present disclosure is similar to the technical problem solved by the control method of bone grinding in the above embodiments, and thus will not be repeated here.

In some embodiments, the positioning apparatus 22 is an optical navigation device, and the optical navigation device is disposed on the grinding drill bit 21. The current position of the grinding drill bit 21 may be collected in real time by using the optical navigation device, and the current position may be reported in real time. The optical navigation device uses a physical optical measurement method to determine a relative position and attitude information by measuring a degree (i.e., speed and distance) of a relative motion between the navigation device and a reference surface. Based on this, the optical navigation device has higher navigation accuracy, i.e., higher accuracy in acquiring the current position of the grinding drill bit 21 in real-time, than a radio navigation device.

In this case, the current position and the positions to be ground are determined based on the pre-established coordinate system. For example, at a beginning of grinding, an optical coordinate system is established based on the optical navigation device, and the current position of the grinding drill bit 21 in the optical coordinate system and each position to be ground in the region to be ground are determined in real time.

For another example, at the beginning of grinding, a reference coordinate system is established based on a base to which the grinding drill bit 21 belongs, and the current position of the grinding drill bit 21 in the optical coordinate system and each position to be ground in the region to be ground are determined in real time.

Here, in order to facilitate an adjustment of the position of the grinding drill bit 21, a connecting member with a plurality of joints is provided between the grinding drill bit 21 and the base, and an angle of each joint can be changed. Based on this, during a grinding process of the grinding drill bit 21, the current position of the grinding drill bit 21 in the reference coordinate system is determined in real time based on the optical navigation device and forward kinematics of a robot. The current position of the grinding drill bit 21 in the reference coordinate system is calculated by using the forward kinematics of the robot. That is, the posture of the robot is obtained by giving a preset angle of each joint, and the current position of the grinding drill bit 21 in the reference coordinate system is calculated.

In some embodiments, the control apparatus 10 is configured to: receive the image data of the portion to be ground from the medical imaging device, and determine the region to be ground corresponding to the portion to be ground and the region boundary of the region to be ground according to the image data; and in response to an operation for indicating a completed grinding by the operator, determine the ground region and send the data of the region to be ground and the data of the ground region.

On this basis, as shown in FIG. 16, the grinding device 20 further includes the display apparatus 23, and the display apparatus 23 is coupled to the control apparatus 10. The display apparatus 23 is configured to receive the data of the region to be ground and the data of the ground region from the control apparatus 10, generate a comparison image of the region to be ground and the ground region, and display the comparison image.

In this case, the generated comparison image is displayed on the display apparatus 23, which enables the operator to know whether the region to be ground is completely ground clearly and intuitively.

As shown in FIG. 17, in some embodiments of the present disclosure, the terminal device 30 is also provided. The terminal device 30 is coupled to the control apparatus 10 in any one of the above embodiments. The control apparatus 10 includes the first communication circuit 14, and the terminal device 30 includes a second communication circuit 31, a main control chip 32 and an alarm 33. The second communication circuit 31 is coupled to the first communication circuit 14. The second communication circuit 31 is configured to receive the prompt control signal from the control apparatus 10. The main control chip 32 is coupled to the second communication circuit 31. The main control chip 32 is configured to, in response to the prompt control signal, control the alarm 33 to send out the prompt signal for prompting the operator that the grinding drill bit 21 (referring to FIG. 15) is adjacent to the region boundary of the region to be ground.

In this case, according to the prompt signal sent by the terminal device 30 for prompting the operator that the grinding drill bit 21 is adjacent to the region boundary, the operator may be well assisted in grinding.

Here, the terminal device 30 may be a mobile phone and/or a Bluetooth bracelet, which is not limited. Here, the bracelet is taken as an example for detailed explanation. For example, the terminal device 30 is the Bluetooth bracelet. In this way, during the grinding process, the operator may wear the Bluetooth bracelet on an arm, so that a status of the terminal device 30 may be monitored in real time without affecting an operation of the operator.

Here, the alarm 33 may be at least one of a speaker, a signal lamp or a vibrator of the terminal device. Accordingly, the prompt signal generated by the alarm 33 may be at least one of vibration generated by the vibrator, sound generated by the speaker or light generated by the signal lamp, which is not limited.

In order to prompt the operator that the grinding drill bit 21 stops operating, in some embodiments, referring to FIG. 17, the second communication circuit is further configured to receive the end control signal from the control apparatus 10. The main control chip 32 is further configured to, in response to the end control signal, control the alarm 33 to send out the prompt signal for prompting the operator that the grinding drill bit 21 (referring to FIG. 15) stops operating.

In this way, when the current position reaches the region boundary and the grinding drill bit 21 stops operating, the terminal device 30 sends out the prompt signal for prompting the operator that the grinding drill bit 21 stops operating, which may promptly remind the operator to perform subsequent operations.

In some embodiments, the control apparatus 10 is further configured to send information of the current position of the grinding drill bit to the terminal device 30 in real time. The second communication circuit 31 is further configured to receive the information of the current position from the control apparatus 10.

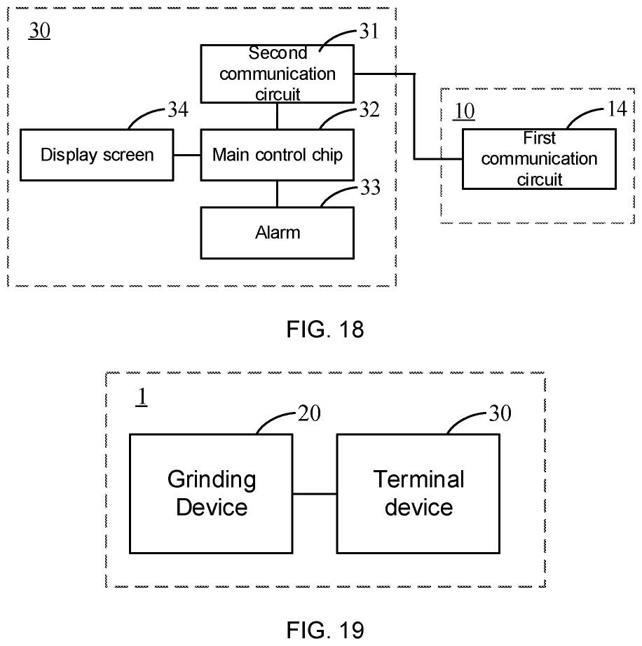

On this basis, as shown in FIG. 18, the terminal device 30 further includes a display screen 34, and the display screen 34 is coupled to the main control chip 32. The display screen 34 is configured to display the current position of the grinding drill bit 21 (referring to FIG. 15) in real time according to the information of the current position under a control of the main control chip 32.

In this case, the terminal device 30 displays the current position of the grinding drill bit 21 in real time according to the received information of the current position, and the operator may know the current position of the grinding drill bit 21 in real time clearly and intuitively, which may well assist the operator in grinding.

As shown in FIG. 19, in some embodiments of the present disclosure, a grinding system 1 is also provided. The grinding system 1 includes the grinding device 20 in any one of the above embodiments and the terminal device 30 in any one of the above embodiments, and the grinding device 20 is coupled to the terminal device 30.

The technical problem solved by the grinding system 1 in some embodiments of the present disclosure is similar to the technical problem solved by the control method of bone grinding in the above embodiments, and thus will not be repeated here.

In yet another aspect, in some embodiments of the present disclosure, a computer-readable storage medium is also provided. The computer-readable storage medium stores computer program instructions. When running on a processor, the computer program instructions enable the processor to execute one or more steps of the control method in any one of the above embodiments.

For example, the computer-readable storage medium may include, but is not limited to, a magnetic storage device (e.g., a hard disk, a floppy disk or a magnetic tape), an optical disk (e.g., a compact disk (CD), a digital versatile disk (DVD)), a smart card and a flash memory device (e.g., an erasable programmable read-only memory (EPROM), a card, a stick or a key driver). Various computer-readable storage media described may represent one or more devices and/or other machine-readable storage media for storing information. The term “machine-readable storage media” may include, but is not limited to, wireless channels and various other media capable of storing, containing and/or carrying instructions and/or data.

In yet another aspect, in some embodiments of the present disclosure, a computer program product is further provided. The computer program product includes computer program instructions. When the computer program instructions are executed on a computer, the computer program instructions enable the computer to execute one or more steps of the control method in any one of the above embodiments.

In yet another aspect, in some embodiments of the present disclosure, a computer program is further provided. When the computer program is executed on a computer, the computer program enables the computer to execute one or more steps of the control method in any one of the above embodiments.

Beneficial effects of the computer-readable storage medium, the computer program product, and the computer program are the same as those of the control method of bone grinding in some embodiments described above, which will not be repeated here.

The foregoing descriptions are merely specific implementation manners of the present disclosure, but the protection scope of the present disclosure is not limited thereto. Changes or replacements that any person skilled in the art could conceive of within the technical scope of the present disclosure shall be included in the protection scope of the present disclosure. Therefore, the protection scope of the present disclosure shall be subject to the protection scope of the claims.

1. A control method of bone grinding, the control method comprising:
receiving a current position of a grinding drill bit from a positioning apparatus in real time;
determining whether the current position reaches a region boundary of a region to be ground in a grinding state; and
controlling the grinding drill bit to stop operating when the current position reaches the region boundary.

2. The control method according to claim 1, wherein determining whether the current position reaches the region boundary of the region to be ground, includes:
selecting a plurality of position points on the region boundary as reference position points;
calculating a distance between each reference position point and the current position; and
determining that the current position reaches the region boundary when there is a distance, between at least one reference position point and the current position, that is substantially zero.

3. The control method according to claim 2, wherein along the region boundary, a distance between every two adjacent reference position points is substantially equal to a set step value.

4. The control method according to claim 2, wherein after calculating the distance between the current position and each reference position point, the control method further comprises:
determining whether there is a distance, between at least one reference position point and the current position, that is less than or equal to a preset critical value; and
if there is the distance, between the at least one reference position point and the current position, that is less than or equal to the preset critical value, sending a prompt control signal to a terminal device, so as to control the terminal device to send out a prompt signal for prompting that the grinding drill bit is adjacent to the region boundary.

5. The control method according to claim 4, wherein there are a plurality of preset critical values, and the plurality of preset critical values are unequal;
determining whether there is the distance, between the at least one reference position point and the current position, that is less than or equal to the preset critical value, and if there is the distance, between the at least one reference position point and the current position, that is less than or equal to the preset critical value, sending a prompt control signal to the terminal device, so as to control the terminal device to send out the prompt signal, include:
performing a plurality of determinations according to the plurality of preset critical values, respectively, each determining process being to determine whether there is a distance, between at least one reference position point and the current position, that is less than or equal to a corresponding preset critical value; and
when a determination result indicates that there is a distance, between at least one reference position point and the current position, that is less than or equal to a corresponding preset critical value, sending a different prompt control signal correspondingly, so as to control the terminal device to send out a different prompt signal.

6. The control method according to claim 5, wherein the plurality of determinations performed respectively according to the plurality of preset critical values are successively performed in a descending order of the plurality of preset critical values.

7. The control method according to claim 4, further comprising:
when there is the distance, between the at least one reference position point and the current position, that is less than or equal to the preset critical value, sending a deceleration control signal to the grinding drill bit, so as to control an operating speed of the grinding drill bit to be decreased.

8. The control method according to claim 5, wherein the plurality of preset critical values include a first preset critical value and a second preset critical value, and the second preset critical value is less than the first preset critical value;
performing the plurality of determinations according to the plurality of preset critical values, respectively; and when the determination result indicates that there is the distance, between the at least one reference position point and the current position, that is less than or equal to the corresponding preset critical value, sending the different prompt control signal correspondingly, include:
determining whether there is a distance, between at least one reference position point and the current position, that is less than or equal to the first preset critical value;
if there is the distance, between the at least one reference position point and the current position, that is less than or equal to the first preset critical value, sending a first prompt control signal to the terminal device, so as to control the terminal device to send out a first prompt signal for prompting that the grinding drill bit is adjacent to the region boundary;
at a next moment, determining whether there is a distance, between at least one reference position point and a current position corresponding to the next moment, that is less than or equal to the second preset critical value; and
if there is the distance, between the at least one reference position point and the current position corresponding to the next moment, that is less than or equal to the second preset critical value, sending a second prompt control signal to the terminal device, so as to control the terminal device to send out a second prompt signal for prompting that the grinding drill bit is adjacent to the region boundary.

9. The control method according to claim 8, further comprising:
when there is the distance, between the at least one reference position point and the current position, that is less than or equal to the first preset critical value, sending a first deceleration control signal to the grinding drill bit, so as to control an operating speed of the grinding drill bit to be decreased to a first preset operating speed; and
when there is the distance, between the at least one reference position point and the current position corresponding to the next moment, that is less than or equal to the second preset critical value, sending a second deceleration control signal to the grinding drill bit, so as to control the operating speed of the grinding drill bit to be decreased to a second preset operating speed,
wherein the second preset operating speed is less than the first preset operating speed.

10. The control method according to claim 9, wherein the plurality of preset critical values further include a third preset critical value, and the third preset critical value is less than the second preset critical value;
performing the plurality of determinations according to the plurality of preset critical values, respectively; and when the determination result indicates that there is the distance, between the at least one reference position point and the current position, that is less than or equal to the corresponding preset critical value, sending the different prompt control signal correspondingly, further include:
when there is the distance, between the at least one reference position point and the current position, that is less than or equal to the second preset critical value, at a next moment, determining whether there is a distance, between at least one reference position point and a current position corresponding to the next moment, is less than or equal to the third preset critical value; and
if there is the distance, between the at least one reference position point and the current position corresponding to the next moment, that is less than or equal to the third preset critical value, sending a third prompt control signal to the terminal device, so as to control the terminal device to send out a third prompt signal for prompting that the grinding drill bit is adjacent to the region boundary.

11. The control method according to claim 10, further comprising: when there is the distance, between the at least one reference position point and the current position corresponding to the next moment, that is less than or equal to the third preset critical value, sending a third deceleration control signal to the grinding drill bit, so as to control the operating speed of the grinding drill bit to be decreased to a third preset operating speed,
wherein the third preset operating speed is less than the second preset operating speed.

12. The control method according to claim 1, wherein after controlling the grinding drill bit to stop operating, the control method further comprises:
determining whether the current position of the grinding drill bit is adjusted back into the region to be ground without being located on the region boundary of the region to be ground;
if the current position of the grinding drill bit is adjusted back into the region to be ground without being located on the region boundary of the region to be ground, sending an in-position control signal to a terminal device, so as to control the terminal device to send out a prompt signal for prompting that the grinding drill bit is adjusted back into the region to be ground; and
in response to an operation for restarting the grinding drill bit, controlling the grinding drill bit to restart.

13. The control method according to claim 1, further comprising:
in response to an operation for indicating a completed grinding, determining a ground region; and
sending data of the region to be ground and data of the determined ground region to a display apparatus, so that the display apparatus generates a comparison image of the region to be ground and the ground region, and displays the comparison image.

14. The control method according to claim 1, wherein before receiving the current position of the grinding drill bit from the positioning apparatus in real time, the control method further comprises:
receiving image data of a portion to be ground from a medical imaging device; and
determining a region to be ground corresponding to the portion to be ground and a region boundary of the region to be ground according to the image data.

15. The control method according to claim 1, wherein before determining whether the current position reaches the region boundary of the region to be ground, the control method further comprises:
determining whether the current position of the grinding drill bit is in the region to be ground;
if the current position of the grinding drill bit is in the region to be ground, controlling the grinding drill bit to start operating.

16-21. (canceled)

22. A terminal device, comprising:
a second communication circuit configured to receive a prompt control signal; and
a main control chip and an alarm coupled to the main control chip, the main control chip being further coupled to the second communication circuit and configured to, in response to a prompt control signal, control the alarm to send out a prompt signal for prompting that the grinding drill bit is adjacent to the region boundary of the region to be ground.

23. The terminal device according to claim 22, wherein
the second communication circuit is further configured to receive information of a current position; and
the terminal device further includes a display screen coupled to the main control chip and configured to display the current position of the grinding drill bit in real time according to the information of the current position under a control of the main control chip.

24. (canceled)

25. A non-transitory computer-readable storage medium storing computer program instructions that, when run on a processor, enable the processor to perform one or more steps of the control method according to claim 1.

26. The control method according to claim 7, wherein there are a plurality of preset critical values, and the plurality of preset critical values are unequal,
determining whether there is the distance, between the at least one reference position point and the current position, that is less than or equal to the preset critical value, and when there is the distance, between the at least one reference position point and the current position, that is less than or equal to the preset critical value, sending the deceleration control signal to the grinding drill bit, so as to control the operating speed of the grinding drill bit to be decreased, include:
performing a plurality of determinations according to the plurality of preset critical values, respectively, each determining process being to determine whether there is a distance, between at least one reference position point and the current position, that is less than or equal to a corresponding preset critical value; and
if a determination result indicates that there is a distance, between the at least one reference position point and the current position, that is less than or equal to a corresponding preset critical value, sending a different deceleration control signal correspondingly, so as to control the operating speed of the grinding drill bit to be decreased to a preset operating speed, wherein
the preset operating speed decreases as the preset critical value decreases.

27. A computer program product, comprising computer program instructions stored in a non-transitory computer-readable storage medium that, when executed on a computer, enable the computer to perform one or more steps of the control method according to claim 1.
